(19) United States
(12) Patent Application Publication
(10) Pub. No.: US 2025/0382254 A1
(43) Pub. Date: Dec. 18, 2025

(54) DIRECT CONVERSION OF GLYCEROL TO ACRYLIC ACID OVER WOx/ZrOx AND MIXED METAL OXIDE

(71) Applicant: Rohm and Haas Company, Collegeville, PA (US)

(72) Inventors: Daniel J. Martenak, Perkasie, PA (US); Christopher D. Frick, Pottstown, PA (US)

(21) Appl. No.: 18/877,195

(22) PCT Filed: Jun. 23, 2023
(86) PCT No.: PCT/US2023/026031

§ 371 Date: Dec. 19, 2024

Related U.S. Application Data
(60) Provisional application No. 63/356,618, filed on Jun. 29, 2022.

(51) Int. Cl. C07C 57/04; B01J 21/06; B01J 23/20; B01J 23/28; B01J 23/30; B01J 27/057; B01J 35/00; C07C 31/22; C07C 45/66; C07C 49/20; C07C 51/25

(52) U.S. Cl. CPC C07C 57/04; B01J 21/066; B01J 23/20; B01J 23/28; B01J 23/30; B01J 27/0576; B01J 35/19; C07C 31/225; C07C 45/66; C07C 49/20; C07C 51/25

(57) ABSTRACT

Acrylic acid is produced by dehydrating glycerol over a first mixed metal oxide catalyst in the presence of oxygen and water to produce acrolein, and then oxidizing the acrolein over a second mixed metal oxide catalyst in the presence of oxygen and water to produce the acrylic acid. The first mixed metal oxide catalyst comprises oxides of tungsten and zirconium. The second mixed metal oxide catalyst comprises a solid catalyst having the empirical formula AₐV_{b}N_{c}X_{d}Oₑ wherein A is at least one element selected from the group consisting of Mo and W, N is at least one element selected from the group consisting of Te and Se, and X is at least one element selected from the group consisting of Nb, Ta, Ti, Al, Zr, Cr, Mn, Fe, Ru, Co, Rh, Ni, Pt, Bi, B, In, Ce, As, Ge, Sn, Li, Na, K, Rb, Cs, Fr, Be, Mg, Ca, Sr, Ba, Ra, Hf, Pb, P, Pm, Eu, Gd, Dy, Ho, Er, Tm, Yb, Lu, Au, Ag, Re, Pr, Zn, Ga, Pd, Ir, Nd, Y, Sm, Tb, Br, Cu, Sc, CI, F and I. A, V, N and X are present in such amounts that the atomic ratio of A:V:N:X is a:b:c:d wherein a=1, b=0.1 to 2, c=0.1 to 1, d=0.01 to 1 and e is dependent on the oxidation state of the other elements in the second mixed metal oxide catalyst.

### FIELD OF THE INVENTION

The present invention relates to a process for the direct conversion of glycerol to acrylic acid.

### BACKGROUND OF THE INVENTION

Various processes for preparing acrylic acid are known in the art. Most commercial acrylic acid is produced using fossil fuel based feedstock, such as, for example, propylene. Starting with propylene, the process for producing acrylic acid is generally conducted in a two-step process in which propylene is converted to acrolein in the first step, followed by the conversion of acrolein to acrylic acid in the second step. This process is very energy intensive, requiring temperatures as high as 360° C. The majority of propylene is a product of fossil fuels.

U.S. Pat. No. 9,546,124 discloses a process for preparing acrylic acid from glycerol by reacting glycerol and a carboxylic to produce allyl alcohol, which is then oxidized to form a mixture of 3-hydroxypropionic acid and acrylic acid.

U.S. Pat. No. 9,409,847 discloses a catalyst for the liquid phase oxydehydration of glycerol to acrylic acid using hydrogen peroxide as an oxidant. This process uses a nanocrystalline copper supported alpha-MnO₂ catalyst.

U.S. Pat. No. 8,748,545 discloses a process for producing acrylic acid from glycerol. Glycerol is subjected to a dehydration reaction carried out over solid acid catalysts.

Although processes for producing acrylic acid from glycerol exist, it would be desirable to produce acrylic acid from renewable materials, such as from biomass-derived feedstocks, that can be easily scaled up to commercial quantities.

### SUMMARY OF THE INVENTION

The present invention is directed to methods for preparing acrylic acid from glycerol.

According to an aspect of the present invention, a method comprises dehydrating glycerol over a first mixed metal oxide catalyst in the presence of oxygen and water to produce acrolein and oxidizing the acrolein over a second mixed metal oxide catalyst in the presence of oxygen and water to produce acrylic acid. The first mixed metal oxide catalyst comprises oxides of tungsten and zirconium. The second mixed metal oxide catalyst comprises a solid catalyst having the empirical formula AₐV_{b}N_{c}X_{d}Oₑ wherein A is at least one element selected from the group consisting of Mo and W, N is at least one element selected from the group consisting of Te and Se, and X is at least one element selected from the group consisting of Nb, Ta, Ti, Al, Zr, Cr, Mn, Fe, Ru, Co, Rh, Ni, Pt, Bi, B, In, Ce, As, Ge, Sn, Li, Na, K, Rb, Cs, Fr, Be, Mg, Ca, Sr, Ba, Ra, Hf, Pb, P, Pm, Eu, Gd, Dy, Ho, Er, Tm, Yb, Lu, Au, Ag, Re, Pr, Zn, Ga, Pd, Ir, Nd, Y, Sm, Tb, Br, Cu, Sc, CI, F and I. A, V, N and X are present in such amounts that the atomic ratio of A:V:N:X is a:b:c:d wherein a=1, b=0.1 to 2, c=0.1 to 1, d=0.01 to 1 and e is dependent on the oxidation state of the other elements in the second mixed metal oxide catalyst.

### DETAILED DESCRIPTION OF EMBODIMENTS OF THE INVENTION

As used herein, the terms “a,” “an,” “the,” “at least one,” and “one or more” are used interchangeably. The terms “comprises,” “includes,” “contains,” and variations thereof do not have a limiting meaning where these terms appear in the description and claims. Thus, for example, a mixture that includes a polymerization inhibitor can be interpreted to mean that the mixture comprises at least one polymerization inhibitor.

As used herein, recitations of numerical ranges by endpoints includes all numbers subsumed in that range (e.g. 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.1 to 100, from 1 to 99.99, from 1.01 to 99.99, from 40 to 6, from 1 to 55, etc.

As used herein, the recitations of numerical ranges and/or numerical values, including such recitations in the claims, can be read to include the term “about.” In such instances, the term “about” refers to numerical ranges and/or numerical values that are substantially the same as those recited herein.

The method of the present invention relates to a method for producing acrylic acid from glycerol.

In the inventive process, glycerol is dehydrated over a first mixed metal oxide catalyst in the presence of oxygen and water to produce acrolein.

The first mixed metal oxide catalyst is a solid catalyst comprising oxides of tungsten (W) and zirconium (Zr). The first mixed metal oxide catalyst may also contain at least one additional element selected from Co, Ni, Mo, Ti, or combinations thereof. When the first mixed metal oxide catalyst contains at least one additional element, the tungsten and zirconium are the main metal elements present. Preferably, the first mixed metal oxide catalyst comprises at least 75 wt. % of tungsten and zirconium based on the total weight of metals in the first mixed metal oxide catalyst, preferably at least 80 wt. %, more preferably at least 85 wt. %, even more preferably at least 90 wt. %, still more preferably at least 95 wt. %, and yet even more preferably at least 98 wt. %. The first mixed metal oxide may comprise 100 wt. % tungsten and zirconium based on the total weight of metals in the first mixed metal oxide catalyst. When Co, Ni, Mo, Ti, or combinations thereof are present in the first mixed metal oxide catalyst, the total amount of Co, Ni, Mo, and Ti may range from 2 to 25 wt. % based on the total weight of metals in the first mixed metal oxide catalyst.

The acrolein produced by the dehydration of glycerol is then oxidized over a second mixed metal oxide catalyst in the presence of oxygen and water to produce acrylic acid.

The second mixed metal oxide catalyst is a solid catalyst having the empirical formula AₐV_{b}N_{c}X_{d}Oₑ wherein A is at least one element selected from the group consisting of Mo and W, N is at least one element selected from the group consisting of Te and Se, and X is at least one element selected from the group consisting of Nb, Ta, Ti, Al, Zr, Cr, Mn, Fe, Ru, Co, Rh, Ni, Pt, Bi, B, In, Ce, As, Ge, Sn, Li, Na, K, Rb, Cs, Fr, Be, Mg, Ca, Sr, Ba, Ra, Hf, Pb, P, Pm, Eu, Gd, Dy, Ho, Er, Tm, Yb, Lu, Au, Ag, Re, Pr, Zn, Ga, Pd, Ir, Nd, Y, Sm, Tb, Br, Cu, Sc, CI, F and I, wherein A, V, N and X are present in such amounts that the atomic ratio of A:V:N:X is a:b:c:d wherein a=1, b=0.1 to 2, c=0.1 to 1, d=0.01 to 1 and e is dependent on the oxidation state of the other elements. Preferably, a=1, b=0.15 to 0.45, c=0.05 to 0.45 and d=0.01 to 0.1.

Preferably, the second mixed metal oxide comprises a solid catalyst having the empirical formula AₐV_{b}N_{c}X_{d}Oₑ wherein A is Mo, N is Te, and X is Nb.

The process may be conducted as a two-part process in which the dehydration of the glycerol is conducted in a first reactor or first portion of a reactor and the oxidation of the acrolein is conducted in a second reactor or second portion of a reactor. Preferably, the reactor comprises a single tube or a plurality of tubes, such as a tube sheet. The reactor or reactors are preferably heated.

Preferably, the process is conducted in a single reactor. More preferably, the process is conducted in a single reactor comprising a catalyst bed selected from a stacked bed or a mixed bed.

In a stacked bed, the first mixed metal oxide catalyst and second mixed metal oxide catalyst are positioned adjacent one another in the reactor, with the first mixed metal oxide catalyst closest to the inlet of the reactor. In this configuration, the glycerol is dehydrated to form acrolein, which then enters the second mixed metal oxide catalyst to form the acrylic acid.

In a mixed bed, the first mixed metal oxide catalyst and the second mixed metal oxide catalyst are mixed together in a single bed. In this configuration, the dehydration and oxidation reactions take place over the entire length of the catalyst bed.

Preferably, the dehydration and oxidation reactions take place in the gas phase. The glycerol can then be condensed and separated from by-products and unreacted glycerol. Preferably, the unreacted glycerol is recycled to the reactor.

In the dehydration of glycerol and subsequent oxidation of the produced acrolein, the oxygen can be present in the form of purified oxygen, oxygen in air, or lattice oxygen of the first and second mixed metal oxide catalysts. Preferably, the oxygen is from air or the lattice oxygen of the first and second mixed metal oxide catalysts. Preferably, no additional oxidant other than purified oxygen, oxygen in air, or lattice oxygen of the first and second mixed metal oxide catalysts is present in the process.

The steps of dehydrating the glycerol and oxidizing the acrolein may be conducted at a temperature ranging from 150 to 450° C., preferably from 200 to 400° C., and even more preferably from 250 to 350° C.

The steps of dehydrating the glycerol and oxidizing the acrolein may be conducted at a pressure ranging from 1 to 5 bar, preferably from 1 to bar, and even more preferably from 1 to 2 bar. Most preferably, the process is conducted at atmospheric pressure.

Purification of the acrylic acid can be achieved by one or more techniques known in the art, such as, for example, absorption using water or an organic solvent, extraction, fractional distillation, or melt crystallization.

Preferably, the glycerol is produced from a biomass-derived feedstock. Because all of the carbon atoms present in the product acrylic acid, any biomass-derived carbon atoms in the glycerol will be present in the acrylic acid. Preferably, at least 90 wt % of the acrylic acid in the product comprises carbon atoms from biomass-derived feedstock, more preferably at least 95 wt %, even more preferably at least 98 wt %, still more preferably at least 99 wt %, and yet more preferably 100 wt %.

### EXAMPLES

The following examples illustrates the present invention but are not intended to limit the scope of the claims.

### Examples 1-5

In Examples 1 to 5, a feed comprising glycerol in water having the composition shown in Table 1 was mixed with 80 sccm of air and 60 sccm of nitrogen and fed to a reactor tube containing a stacked catalyst bed at 280° C. and atmospheric pressure. The stacked catalyst bed comprised 4.0 cm³ of 14/20 mesh WOₓ/ZrOₓ catalyst atop 3.6 cm³ of 14/20 mesh MoₐV_{b}Nb_{c}Te_{d}O where a=1, b=0.15 to 0.45, c=0.05 to 0.45 and d=0.01 to 0.1. The reactor effluent was condensed in several traps immersed in an isopropanol/dry ice bath. The gaseous product downstream of the traps was analyzed on an SRI gas chromatograph. Condensed liquid products were analyzed on a HP 6890 gas chromatograph.

The reactor consisted of a ½ “o.d., 0.035” wall 316 stainless steel tube, 18″ long, with Swagelok fittings on each end, which permitted connection of the reactor tube to the system. Each reactor was supplied with feed via a bank of Brooks mass flow controllers and a KD Scientific syringe pump or LC pump. Air and nitrogen were fed to the reactor via the mass flow controllers, and the glycerol/water solution was fed via the KD Scientific LC pump. The catalyst samples were diluted 1:1 in quartz chips and charged to the center section (or upper and lower center section) of the ½″ o.d. reactor. The resultant mixed bed was supported below by quartz chips. After the catalyst/quartz chip mixture had been charged, the remaining reactor volume was filled with quartz chips, which acted as a mixing and pre-heat zone. Inlet and outlet fittings were affixed to the reactor tube, which was then subjected to a 20 psig pressure test on the lab bench prior to mounting in the reactor system. The reactor tube containing the catalyst was mounted in the furnace and heavily insulated to insure adequate heat transfer.

Once the reactor tube was mounted in each reactor system, the system was isolated and pressure tested with nitrogen at 100 psig. If a reactor system did not maintain pressure, the system was de-pressurized (following a prescribed procedure), and all reactor fittings were tightened. The reactor system was then re-tested. This process continued until the reactor system was leak-tight at 100 psig. Following pressure testing, the reactor was swept by nitrogen to remove residual air, and the furnace temperature was increased to the initial test condition. Once the reactor had reached the desired temperature in flowing nitrogen, the syringe pump was started to initiate liquid feed flow, and air flow was initiated at the desired flow rate. The back pressure regulator, if required, was adjusted to maintain the desired reactor pressure. Once full feed flow had been established, the reactor was lined out for ˜45 minutes before initiating an experiment. Reaction temperatures ranged from 250-350° C. All experiments were conducted at atmospheric pressure.

### Comparative Examples 1 and 2

Comparative Examples 1 and 2 were conducted in a similar manner as Examples 1 to 5 with the exception that a single mixed metal catalyst was used. A feed comprised of 20.97 wt % glycerol in water was mixed with 80 sccm of air and 60 sccm of nitrogen and fed to the reactor tube containing a single bed of 3.6 cm³ of 14/20 mesh MoVNbTeO catalyst, as used in Examples 1-5, at 280° C. and atmospheric pressure. The reactor effluent was condensed in several traps immersed in an isopropanol/dry ice bath. The gaseous product down stream of the traps was analyzed on an SRI GC. Condensed liquid products were analyzed on a HP 6890 GC.

TABLE 1

| | | | Rx. | Peak | Glycerol | Product Selectivities | | | |
|---|---|---|---|---|---|---|---|---|---|
| | | | Temp. | Temp. | Conv | | Acetic | Propionic | Acrylic |
| Example | Catalyst | Feed | ° C. | ° C. | by Diff. % | Acrolein | Acid | Acid | Acid |
| Example 1 | WOx/ZrOx | 21.87% glycerol/H2O | 280 | 274 | 96.81 | 0.22 | 9.10 | 0.75 | 70.80 |
| | MoVNbTeO | | 270 | 310 | | | | | |
| Example 2 | WOx/ZrOx | 21.87% glycerol/H2O | 290 | 282 | 100 | 0.08 | 9.40 | 0.54 | 70.97 |
| | MoVNbTeO | | 280 | 312 | | | | | |
| Example 3 | WOx/ZrOx | 21.87% glycerol/H2O | 305 | 296 | 100 | 0.22 | 10.64 | 0.47 | 73.00 |
| | MoVNbTeO | | 295 | 325 | | | | | |
| Example 4 | WOx/ZrOx | 21.87% glycerol/H2O | 315 | 308 | 100 | 0.16 | 10.57 | 0.33 | 68.28 |
| | MoVNbTeO | | 305 | 343 | | | | | |
| Example 5 | WOx/ZrOx | 39.91% glycerol/H2O | 290 | 282 | 100 | 0.73 | 12.35 | 0.52 | 61.98 |
| | MoVNbTeO | | 280 | 365 | | | | | |
| Comp. Ex. | MoVNbTeO | 20.97% glycerol/H2O | 280 | 350 | 100 | 0 | 14.24 | 0.30 | 17.11 |
| 1 | | | | | | | | | |
| Comp. Ex. | MoVNbTeO | 20.97% glycerol/H2O | 250 | 322 | 100 | 0.17 | 14.10 | 0.53 | 16.22 |
| 2 | | | | | | | | | |

We claim:

1. A method comprising:
dehydrating glycerol over a first mixed metal oxide catalyst in the presence of oxygen and water to produce acrolein,
oxidizing the acrolein over a second mixed metal oxide catalyst in the presence of oxygen and water to produce acrylic acid,
wherein the first mixed metal oxide catalyst comprises oxides of tungsten and zirconium, and
wherein the second mixed metal oxide catalyst comprises a solid catalyst having the empirical formula: 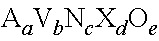
wherein:
A is at least one element selected from the group consisting of Mo and W,
N is at least one element selected from the group consisting of Te and Se, and
X is at least one element selected from the group consisting of Nb, Ta, Ti, Al, Zr, Cr, Mn, Fe, Ru, Co, Rh, Ni, Pt, Bi, B, In, Ce, As, Ge, Sn, Li, Na, K, Rb, Cs, Fr, Be, Mg, Ca, Sr, Ba, Ra, Hf, Pb, P, Pm, Eu, Gd, Dy, Ho, Er, Tm, Yb, Lu, Au, Ag, Re, Pr, Zn, Ga, Pd, Ir, Nd, Y, Sm, Tb, Br, Cu, Sc, CI, F and I,
wherein A, V, N and X are present in such amounts that the atomic ratio of A:V:N:X is a:b:c:d wherein a=1, b=0.1 to 2, c=0.1 to 1, d=0.01 to 1 and e is dependent on the oxidation state of the other elements in the second mixed metal oxide catalyst.

2. The method according to claim 1, wherein the steps of dehydrating the glycerol and oxidizing the acrolein are performed in a single reactor.

3. The method according to claim 2, wherein the single reactor comprises the first and second mixed metal oxide catalysts in a single bed selected from a stacked bed and a mixed bed.

4. The method according to claim 3, wherein the single reactor comprises the first and second mixed metal oxide catalysts in a stacked bed.

5. The method according to claim 1, wherein the oxygen is present in the form of purified oxygen, air, or lattice oxygen of the mixed metal oxide.

6. The method according to claim 1, wherein the process is conducted in the gas phase.

7. The method according to claim 1, wherein the oxygen in the steps of dehydrating the glycerol and oxidizing the acrolein and/or the lattice oxygen of the first and second mixed metal oxide catalysts are the only oxidants present in the process.

8. The method according to claim 1, wherein the acrylic acid is produced from biomass-derived feedstock.

9. The method according to claim 8, wherein at least 90% of the carbon atoms in the acrylic acid are derived from biomass-derived feedstock.

10. The method according to claim 1, wherein A is Mo, N is Te, and X is Nb.

11. The method according to claim 1, a=1, b=0.15 to 0.45, c=0.05 to 0.45 and d=0.01 to 0.1.

12. The method according to claim 1, wherein the steps of dehydrating the glycerol and oxidizing the acrolein are conducted at a pressure ranging from 1 to 5 bars.

13. The method according to claim 1, wherein the steps of dehydrating the glycerol and oxidizing the acrolein are conducted at a temperature ranging from 150 to 450° C.

14. The method according to claim 1, wherein the steps of dehydrating the glycerol and oxidizing the acrolein are conducted in a single step.

15. The method according to claim 1, further comprising recycling unreacted glycerol to the dehydrating step.
